(19) United States
(12) Patent Application Publication
(10) Pub. No.: US 2026/0102336 A1
(43) Pub. Date: Apr. 16, 2026

(54) Veterinary Soft-Chewable Product and Process for the Manufacture Thereof

(71) Applicant: VIRBAC, Carros (FR)

(72) Inventors: David CORVAISIER, Biot (FR); Béatrice SCORTECCI SALEIX, Nice (FR)

(73) Assignee: VIRBAC, Carros (FR)

(21) Appl. No.: 19/113,811

(22) PCT Filed: Jul. 20, 2023
(86) PCT No.: PCT/EP2023/070230

§ 371 Date: Mar. 20, 2025

(30) Foreign Application Priority Data
Jul. 27, 2022 (FR) FR2207711

(51) Int. Cl. A61K 9/00; A61K 31/5415; A61K 47/02; A61K 47/10; A61K 47/36; A61K 47/38; A61K 47/44; A61K 47/46

(52) U.S. Cl. CPC A61K 9/0056; A61K 31/5415; A61K 47/02; A61K 47/10; A61K 47/36; A61K 47/38; A61K 47/44; A61K 47/46

(57) ABSTRACT

A soft-chewable product (1) for oral administration, having at least one pair of score markings (5, 6) configured to favor a division of the product into doses (1a, 1b), the force required to divide the product (1) into doses (1a, 1b) along the at least one pair of score markings (5, 6) being between 10 and 60 N and the loss of mass during the division of the product (1) into doses (1a, 1b) being less than 0.5%.

### TECHNICAL FIELD OF THE INVENTION

The invention relates to a product in soft-chewable form, in particular a soft-chew, which can be easily divided into several doses by application of a moderate manual force. The subject of the invention is more specifically such a product for veterinary use, in particular for dogs and cats, as well as a process for its manufacture.

### TECHNICAL BACKGROUND

In the veterinary field, the oral route is the easiest and most convenient way to administer a medication to an animal, at least for their owners. Numerous types of dosage forms for the oral route have been developed, including solid dosage forms, among which tablets are the most common.

Hard tablets and chewable tablets are made from conventional dusty, granular, and pulverulent materials. Their dry and gritty texture makes them often unappealing to many animals. In addition, hard and chewable tablets crumble in the mouth of the animal upon chewing and it is frequent that part of the tablet falls outside of the animal's mouth. Compliance with the dosage is thereby compromised.

On the contrary, soft-chewable products have a soft texture, a pleasant mouth feel, and an agreeable taste to the animals and they are generally easily accepted by the animal. In addition, soft-chewable tablets are moist and maintain sufficient cohesiveness when chewed by the animal and they do not crumble, in sharp contrast with hard tablets and chewable tablets.

Most often, soft-chewable products are unitary dosage of a defined form and weight, including the same amount of active substances. They are almost exclusively obtained by compression, molding or extrusion of a paste containing the active substance(s).

Insofar as the quantity of active substances to be administered can vary from one subject to another, in particular according to the weight of the subject, animal health companies have developed products of different sizes intended for more or less broad weight ranges. For example, some veterinary products are sold as tablets indicated for dogs weighing between 20 and 40 kg. The administration of the tablet to a 20 kg dog may result in an over-dosage and cause adverse effects while the administration of the same tablet to a 40 kg dog may result in an under-dosage and impair the efficacy of the treatment.

However, having products for more reduced weight ranges is limited by practical and economic considerations, e.g. the need to multiply the numbers of manufacturing campaigns of the same product but in different dosage strengths and the number of product presentations.

Products made of a soft, divisible chewable composition have been described in prior art. US20140141055 describes the possibility of adding on one face of a soft-chew obtained by molding a score, a cross score or a score marking. US2020360290 describes “soft tablets” having a score marking to divide the tablet in up to 4 doses.

Besides, it has been described in prior art to introduce score markings facing each other on opposite sides of hard tablets to facilitate their division. Soft chews and hard tablets however differ significantly in their rheological properties: solid tablets are designed to be hard, brittle and resistant to breaking while soft chews have a soft (ductile), deformable and plastic texture.

It is known that the rupture (or fracture) of a material into two or more parts under the action of a stress occurs differently depending on whether it is a brittle material or a deformable material. For a brittle material such as a tablet, the rupture occurs by a mechanism called brittle fracture: when a stress is exerted on the material, said material does not deform and the rupture occurs when the applied stress reaches the maximum value which he can resist (ultimate resistance). The rupture is the result of the formation of a so-called unstable crack and its spontaneous and extremely rapid propagation. The fracture surface is usually clean. Reducing the thickness of the tablet with a score marking provides a predetermined weak point which lowers the ultimate resistance and allows the formation of the crack with a lesser stress. By inserting a second score marking opposite the first, a predetermined fracture path is created which guides the direction of the crack during propagation. The fracture surface, intrinsically clean, then extends from one groove to the other.

For a deformable material such as a soft-chew, failure occurs by a mechanism known as ductile failure: unlike a brittle material, the material deforms under the effect of stress but failure does not occur when the ultimate resistance is reached. Failure occurs when the material has reached its plastic limit. The material does not literally break but tears as the result of the formation of a stable crack which does not propagate spontaneously but under the effect of an additional stress. The fracture surface is usually irregular due to the deformation that the material underwent before failure. With ductile products, such as soft chews, a score marking can facilitate the division by lowering the stress required to initiate the crack. However, the crack, once initiated, does not propagate spontaneously.

### SUMMARY OF THE INVENTION

The object of the invention is to solve the problems associated with the divisibility of soft-chewable products of the prior art by improving the precision of the division and the precision of the doses obtained after division, while reducing the risks of contamination of the user by the active ingredients contained in the soft-chewable composition.

It has been found that a soft-chewable product can be more easily divided into doses by carefully selecting the rheological properties of the product and the arrangement of the score markings on the product.

The invention therefore proposes a soft-chewable product for oral administration, comprising a pharmaceutical, veterinary or nutraceutical active substance, a humectant and a forming agent, said soft-chewable product having a pair of score markings facing each other on opposite faces of the product, the strength necessary to divide the product into doses along said pair of score markings being preferably between 10 and 60 N and the loss of mass during the division of the product into doses being less than 0.5%. Preferably, the cumulative depth of the two score markings is greater than 10% of a thickness of the product.

By “soft-chewable product” is meant a dimensionally stable pharmaceutical dosage unit having a flexible and elastic (ductile) texture. A soft-chewable product has a higher moisture content than a conventional tablet or a chewable tablet. Unlike chewable or hard tablets, a soft-chewable product can be deformed without breaking. In other words, if it falls, it does not break but can eventually deform. In the present invention, soft-chewable products are preferably soft chews intended for animals, such as dogs and cats.

Thanks to the physical attributes of the soft-chewable product according to the invention, e.g. configuration of the score markings, a homogeneous division of the product along said score marking(s) is favored, by simple application of a moderate force. The risk of contamination of the person dividing the product is also limited. It is also possible to obtain halves, quarters, sixths or eighths or any fraction of the product, each fraction having a controlled quantity of active substance by adjusting the number of score markings.

In order to provide a soft-chewable product that can be easily divided without loss of mass, other properties, such as firmness and friability of the soft chew should be taken into account as they impact the texture of the soft chew and has an effect on the mass loss during the division of the soft-chewable product. The friability of a soft chew refers to its tendency to crumble or break apart easily when subjected to pressure or handling. It is a measure of the structural integrity and resistance to crumbling of the soft chew. The lower the friability, the less likely the soft-chewable product is to break or crumble, while higher friability indicates a greater tendency to crumble or disintegrate.

In other words, an object of the invention is to provide a soft-chewable product which can be divided, if necessary, into two or more parts comprising a defined and reproducible quantity of substance. The division of the product allows to easily adapt the quantity of substance to be administered according to the dosage and/or the weight of the subject. The pair of score markings configured to favor a division of the product into doses can delimit portions of the product of unequal sizes and weights or portions of the product of identical sizes and weights.

Since it is possible to administer the entire product, any fraction or any multiple of fraction, for example ⅛th, 2/8th, ⅜th, 4/8th, ⅝th, 6/8th, ⅞th or 8/8th when the product can be divided into 8 doses, the product according to the invention has, among other advantages, the following advantages:
a precise quantity of the substance can be administered to subjects whose weight varies widely with the same product. For example, if each dose of the product contains the amount of substance to be administered per 10 kg of subject weight, a 30 kg subject will receive ⅜ of the product and a 70 kg subject will receive ⅞ of the product. This avoids overdosing or underdosing the subject.
the same product can be administered in several successive doses when the dosage requires administration in several times. For example, when the product is to be administered twice a day, the product can be cut in half and each dose taken at the indicated time of the day.
the same product can be administered several times when the subject has difficulty swallowing the product in a single dose. For example, when the subject is geriatric or pediatric, the product can be cut into smaller doses that are easy for the subject to swallow.
the same product can be administered to several subjects at the same time. This is the case when several subjects must receive the same treatment at the same time, for example in a hospital, clinic or veterinary clinic.

It should also be noted that the product according to the invention makes it possible to increase the exchange surface of the undivided product thanks to the presence of the score marking or of the plurality of score markings used. Such a configuration promotes the diffusion of the composition in the body and, consequently, its therapeutic effectiveness when the product is used without being divided.

Another advantage lies in the fact that the same product, which can be used in several doses, makes it possible to reduce the amount of packaging used. For example, in the case of a product to be administered twice daily, the product may be packaged in a single blister instead of two blisters.

According to the features of the invention, which may be implemented independently of each other or according to all technically possible combinations and which form as many variants of the invention:
the force required to divide the product along the score marking is between 20 and 55 N,
the loss of mass during division is less than 0.1% of the total mass of the product,
the score marking has a V section,
the at least one score marking extends parallel to a larger dimension of the product,
said product is formed by extrusion, compression or molding,
cutting starters are provided on at least one score marking,
said product has a firmness of between 10 and 300 N, preferably 10 and 150 N, more preferably between 30 and 80 N,
said product has a friability of less than 1%, preferably 0.5% and more preferably less than 0.2%,
each score markings present in pairs and facing each other on opposite sides has, independently of the other, a depth of between 1/20 and 4/10 of the thickness of the product,
the cumulative depth of the two score markings of one and the same of said pair or pairs is greater than 20%, preferably greater than 30%, more preferably greater than 40%, and in a specific embodiment greater than 50%.
the two score markings of one and the same of said pair or pairs have an identical depth, each of the score markings having a depth of between 5% and 45%, preferably between 10% and 45% and more preferably between 25% and 35%, of a thickness of the product,
the score markings, called first score marking, extend parallel, in particular to a larger dimension of the product,
all said score markings extend parallel to the longest dimension of the product
as a variant, some other score marking, called additional score marking, extend transversely to the largest dimension of the product,
said score marking or pairs of score markings form a grid on the surface of the product,
the score markings are configured to allow the product to be divided into four, six or eight doses,
the score markings are configured to allow division into identical doses,
said product has a cylindrical, elliptical, oblong form or a cuboid and/or parallelepiped form,
the product has a base and protrusions, extending from said base and separated by score markings,
the base has a substantially rectangular cross section,
said product has a mass of between 100 mg and 20 g,
said pharmaceutical, veterinary or nutraceutical active substance is evenly distributed in the product,
said product further comprises excipients,
said product is intended for use as a drug to be administered to a subject, for human or veterinary applications,
said product is intended to be administered in several doses, in particular for a single and same treatment,
said product is intended to be administered at defined and/or regular intervals, each dose providing the subject to be treated with the quantity of effective pharmaceutical, veterinary or nutraceutical active substance until the administration of the following dose,
a dose of the product is intended to be administered several times a day, several days a week and/or several days a month,
said product is intended to be administered to a geriatric subject or to a pediatric subject,
said product is intended for a non-human mammal, preferably a pet,
said product is intended for a dog or a cat, preferably a dog.

The invention also proposes a process for manufacturing a formed soft-chewable product as described above.

Preferably, said method comprises the steps of:
(a) obtaining a composition,
(b) forming the composition to obtain the formed soft-chewable product.

According to a first embodiment, the forming of the composition takes place by compression or by molding, the at least one score marking being hollowed in step (b), in particular by means of a mold of appropriate shape.

According to another embodiment, the forming takes place by extrusion, the at least one score marking being hollowed in step (b), in particular by means of a die of appropriate shape.

### DESCRIPTION OF DRAWINGS

The invention will be better understood, and other aims, details, characteristics and advantages thereof will appear more clearly from the detailed explanatory description which follows, of at least one embodiment of the invention given herein, by way of purely illustrative and non-limiting example, with reference to the appended schematic drawings, including:

FIG. 1 schematically illustrates in perspective a first embodiment of the product according to the invention;

FIG. 2 schematically illustrates in perspective a second embodiment of the product according to the invention;

FIG. 3 schematically illustrates in perspective a third embodiment of the product according to the invention;

FIG. 4 schematically illustrates in elevation a first test performed on a product according to FIG. 1;

FIG. 5 schematically illustrates in elevation a second test carried out on a product according to FIG. 1.

FIG. 6 shows soft-chews having a high friability after an attempt to divide them by hand.

In these figures, the same reference corresponds to identical or similar elements.

### DETAILED DESCRIPTION OF THE INVENTION

The present invention concerns a soft-chewable product 1 for oral administration, comprising a pharmaceutical, veterinary or nutraceutical active substance, a humectant and a forming agent, wherein the product 1 presents a pair of score markings 5, 6 facing each other on opposite faces 3a, 3b of the product and configured for a division of the product into doses 1a, 1b, the cumulative depth of the pair of score markings 5, 6 being greater than 10% of a thickness of the product 1, and the loss of mass during the division of the product 1 into doses 1a, 1b is less than 0.5%.

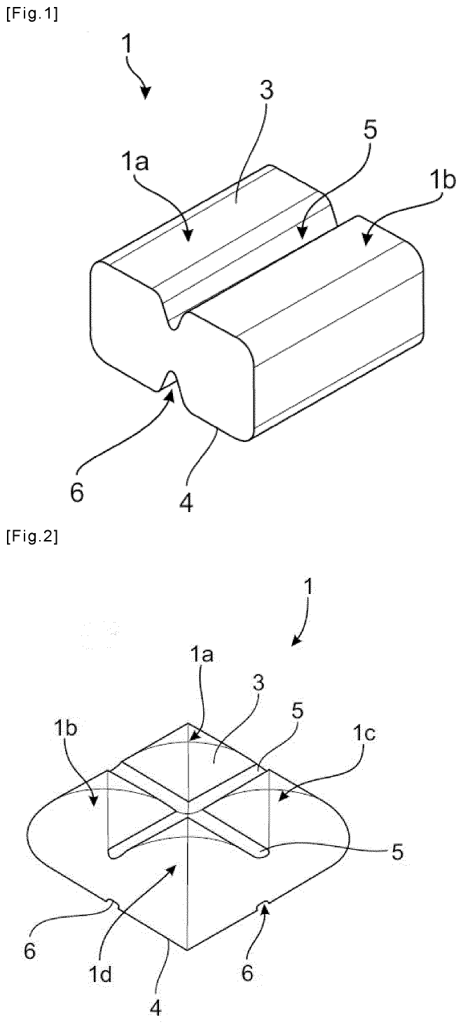

As illustrated in FIGS. 1 to 3, the invention provides a soft-chewable product 1 for oral administration. Said product has at least one score marking 5, 6 configured to favor a division of the product into doses 1a, 1b, 1c, 1d.

The latter is shown before division. To proceed with the division of product 1, the user will be able to grasp two different areas of the product in one of his hands, between the thumb and the index finger. He will then separate them along the at least one score marking 5, 6.

According to the invention, to obtain a controlled and homogeneous manual division of the product, the product has score marking(s) 5, 6 configured so that the force necessary to divide the product into doses along said score marking(s) 5, 6 is between 10 and 60 N, and the mass loss when dividing the product into doses is less than 0.5%. Thanks to such characteristics, each dose includes a controlled amount of substances. In addition, the risks of user contamination are reduced.

The soft-chewable products according to the invention are characterized by their flexible (ductile) texture, softness and susceptibility to elastic deformation. The products according to the invention are also characterized by their texture, in particular the firmness of the product preferably between 10 and 300 N. Unlike solid tablets (e.g. chewable and hard tablets), the products according to the invention can be deformed without breaking.

Unlike tablets, soft-chewable are pliable and they lack the structural integrity necessary for a clean division; they tend to deform and stretch rather than break cleanly.

The firmness corresponds to the force necessary to obtain a given deformation of the product.

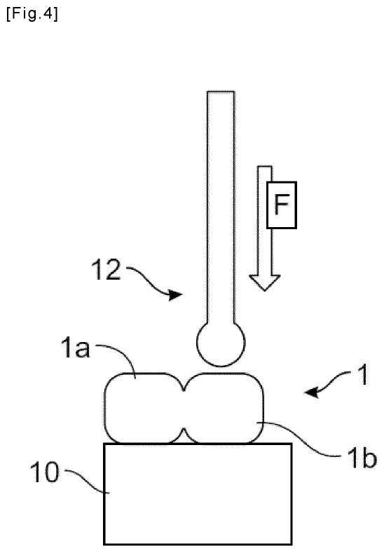

As illustrated in FIG. 4, in the present invention, the firmness is measured on a texturometer, in particular TA XT2i (Stable Micro Systems), at a temperature between 18 and 25° C., using a spherical probe of 8 mm in diameter with the following parameters:

### Compression in Test Mode

Pre-test speed 1.00 mm/sec
Test speed 1.00 mm/sec
Post-test speed 10.00 mm/sec
Target mode distance
Distance 2.00 mm
Trigger Type Auto (Force)
Release force 0.5 N
Number of points per second: 25

These parameters are, for example, implemented directly in the texturometer software.

Briefly, product 1 serving as a sample for the test was placed on a flat base/surface 10 and a spherical probe 12 was lowered into the sample at a distance of 2 mm at a predetermined speed. Sample 1 was deformed by probe 12 and the resistance offered by the sample, corresponding to the force necessary to penetrate 2 mm, was recorded. The resistance measured expressed in newtons (N) corresponds to the firmness of the product. The test is carried out on sample 1 at a distance from score marking(s) 5, 6.

Advantageously, the products according to the invention have a firmness of between 10 and 150 N, preferably between 30 and 80 N.

The composition of the products according to the invention can vary in large proportions. Appropriate compositions for obtaining products having the textural characteristics are for example described in applications WO2005/013714, WO2022/049149, WO2021/180603, WO2016/073347, WO2009/064859, WO2014/079825, WO2007/067582, WO2020/1722 32, WO2021/013825, WO2013/119442, WO2013/150052, WO2021/046296, WO2019/021191, WO2014/141223 and WO2012/049156.

The soft-chewable products according to the invention comprise a pharmaceutical, veterinary or nutraceutical active substance; a humectant and a forming agent.

As used herein, a “humectant” is a substance having the effect of maintaining the softness of the soft-chew over the shelf life of the product. Preferably, the humectant is selected from the group consisting in propylene glycol, glyceryl triacetate, vinyl alcohol, neoagarobiose, sugar polyols such as glycerol, sorbitol, xylitol and maltitol, polymeric polyols such as polydextrose, caprylic/capric triglyceride, propylene glycol dicaprilate/dicarprate, medium chain triglycerides sold under the trademark Miglyol, especially Miglyol 812 or 814, vegetable oils, or mixtures thereof.

As used herein, a “forming agent” is a substance providing texture to the soft-chewable product.

Preferably, the forming agent is selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline cetyl wax, alcohol, polysaccharides, hydroxypropylmethylcellulose, poly(meth)acrylates, alkyl poly(meth)acrylates, and mixtures thereof.

Preferably, the soft-chewable product further comprises a filler (or diluent).

As used herein, a “filler” refers to food-stuffs containing a preponderance of starch and/or starch-like material.

Preferably, the filler is selected from the group consisting in starch such as corn starch, rice starch or potato starch, sucrose, confectioner's sugar, lactose including anhydrous lactose, hydrated lactose, sprayed dried lactose, dextrin, dextrate, mannitol, soy grits, soy protein fines microcrystalline cellulose, silicified microcrystalline cellulose, calcium phosphate, crystalline maltose, maltodextrins, and mixtures thereof. More preferably, the filler is selected from the group consisting in corn starch, lactose, microcrystalline cellulose, confectioner's sugar, and mixtures thereof.

Preferably, the soft-chewable product further contains a palatant.

As used herein, a “palatant” means a non-active ingredient that entices a pet to consume the veterinary dosage form of the invention. Palatants to be used in dosage forms of the present invention may take the form of dry powder palatants, non-powder palatants, or as systems that use both dry powder and non-powder palatants.

Palatants include chicken, turkey, beef, pork, lamb, fish, egg, cheese, seafood, vegetable and vegetable matter, yeast (e.g., brewer's yeast), yeast extracts, and mixtures thereof. Natural meat palatants can be obtained from meat, meat products, organ meat, yeast extracts, vegetable matter, and mixtures thereof. For example, an oral veterinary composition medication might include animal product-based flavorings such as dried or powdered meat and meat parts such as beef, pork, chicken, turkey, fish, and lamb; organ meats such as liver and kidney; meat meals, bone meals and ground bone; and animal-derived food such as casein, milk (which may include dry forms and lowered fat forms, such as dry skim milk), yogurt, gelatin, cheese and egg (collectively, “animal origin palatants”) may be utilized. Non-animal origin palatants include essential oil, oleoresin, essence or extract, protein hydrolysate, distillate, or any product of roasting, heating or enzymolysis, which contains the flavoring constituents derived from a spice, fruit or fruit juice, vegetable or vegetable juice, edible yeast (active and inactive), herb, bark, bud, root, leaf or any other edible portions of a plant. The determination of the type and amount of palatant is easily determined by a practitioner of this art. More preferably, the palatant is selected from the group consisting in animal product-based palatants such as dried or powdered meat and meat parts such as beef, pork, chicken, turkey, fish, and lamb; organ meats such as liver and kidney; meat meals, bone meals and ground bone and non-animal product based palatants such as yeast, yeast extracts and glutamate. Most preferred palatants are chicken (or poultry) liver powder, yeast, yeast extracts, and mixtures thereof.

In a preferred embodiment, the soft-chewable product comprises:
a pharmaceutical, veterinary or nutraceutical active substance,
a humectant selected from the group consisting in propylene glycol, glyceryl triacetate, vinyl alcohol, neoagarobiose, sugar polyols such as glycerol, sorbitol, xylitol and maltitol, polymeric polyols such as polydextrose, caprylic/capric triglyceride, propylene glycol dicaprilate/dicarprate, medium chain triglycerides sold under the trademark Miglyol, especially Miglyol 812 or 814, vegetable oils, or mixtures thereof,
a forming agent selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline wax, cetyl alcohol, polysaccharides, hydroxypropylmethylcellulose, poly(meth)acrylates, alkyl poly(meth)acrylates, and mixtures thereof,
a filler selected from the group consisting in starch such as corn starch, rice starch or potato starch, sucrose, confectioner's sugar, lactose including anhydrous lactose, hydrated lactose, sprayed dried lactose, dextrin, dextrate, mannitol, soy grits, soy protein fines microcrystalline cellulose, silicified microcrystalline cellulose, calcium phosphate, crystalline maltose, maltodextrins and mixtures thereof,
a palatant selected from the group consisting in animal product-based palatants such as dried or powdered meat and meat parts such as beef, pork, chicken, turkey, fish, and lamb; organ meats such as liver and kidney; meat meals, bone meals and ground bone and non-animal product based palatants such as essential oil, oleoresin, essence or extract, protein hydrolysate, distillate, or any product of roasting, heating or enzymolysis, which contains the flavoring constituents derived from a spice, fruit or fruit juice, vegetable or vegetable juice, edible yeast (active and inactive), herb, bark, bud, root, leaf or any other edible portions of a plant, and mixtures thereof.

Of note is a soft-chewable product comprising, based on the total weight of the soft-chewable product:
0.01 to 30%, preferably 0.1 to 20%, more preferably 5 to 15% of a pharmaceutical, veterinary or nutraceutical active substance,
5 to 40%, preferably 10 to 30%, more preferably 10 to 20% of a humectant, preferably selected from the group consisting in propylene glycol, glyceryl triacetate, vinyl alcohol, neoagarobiose, sugar polyols such as glycerol, sorbitol, xylitol and maltitol, polymeric polyols such as polydextrose, caprylic/capric triglyceride, propylene glycol dicaprilate/dicarprate, medium chain triglycerides sold under the trademark Miglyol, especially Miglyol 812 or 814, vegetable oils, or mixtures thereof,
1 to 40%, preferably 1 to 20%, more preferably 10 to 20% of a forming agent, preferably selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline wax, cetyl alcohol, polysaccharides, hydroxypropylmethylcellulose, poly(meth)acrylates, alkyl poly(meth)acrylates, and mixtures thereof,
10 to 60%, preferably 15 to 45%, more preferably 15 to 35% of a filler, preferably selected from the group consisting in starch such as corn starch, rice starch or potato starch, sucrose, confectioner's sugar, lactose including anhydrous lactose, hydrated lactose, sprayed dried lactose, dextrin, dextrate, mannitol, soy grits, soy protein fines microcrystalline cellulose, silicified microcrystalline cellulose, calcium phosphate, crystalline maltose, maltodextrins and mixtures thereof,
10 to 50%, preferably 10 to 35%, more preferably 15 to 35% of a palatant, preferably selected from the group consisting in animal product-based palatants such as dried or powdered meat and meat parts such as beef, pork, chicken, turkey, fish, and lamb; organ meats such as liver and kidney; meat meals, bone meals and ground bone and non-animal product based palatants such as essential oil, oleoresin, essence or extract, protein hydrolysate, distillate, or any product of roasting, heating or enzymolysis, which contains the flavoring constituents derived from a spice, fruit or fruit juice, vegetable or vegetable juice, edible yeast (active and inactive), herb, bark, bud, root, leaf or any other edible portions of a plant, and mixtures thereof, more preferably selected from chicken (or poultry) liver powder, yeast, yeast extracts, and mixtures thereof,
0.1 to 20%, preferably 1 to 15%, more preferably 5 to 15% of a disintegrant, preferably selected from the group consisting in sodium starch glycolate, crospovidone, croscarmellose, and mixtures thereof.

The soft-chewable product as described above may comprise at least one further excipient selected from the group consisting in lubricants, glidants, surfactants and dyes.

By way of non-limiting example, a soft-chewable product comprising a pharmaceutical, veterinary or nutraceutical active substance according to the invention may comprise the following ingredients, by weight of the total weight of the product:
from 1 to 20% of a forming agent, preferably selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline wax, cetyl alcohol, polysaccharides, hydroxypropylmethylcellulose, poly(meth)acrylates, alkyl poly(meth)acrylates, and mixtures thereof,
from 15 to 35% of a filler, for example microcrystalline cellulose, starch or a mixture of these two fillers,
10 to 20% of a humectant, for example a polyethylene glycol, glycerol or a mixture of these two humectants,
from 15 to 35% of at least one palatable agent, for example poultry liver powder or palatants of non-animal origin sold by Diana Petfood,
from 5 to 15% of a disintegrating agent, for example croscarmellose sodium,
optionally excipients chosen from antioxidants, lubricants and flavorings.

The configuration of the score markings can be chosen in a variety of ways insofar as it is configured to allow the division of the product into doses. For example, a product with two orthogonal score markings on opposite surfaces will not be considered as having a configuration favoring the division of the product into doses.

Preferably, the score markings are hollowed out in the thickness of the product. The thickness of the product means the dimension extending perpendicular to the plane of a major lower or upper face of the product, in an oblong, cuboid or parallelepiped configuration, or, where the product has a cylindrical or elliptical form of the longest dimension. In the present invention, when reference is made to a fraction or portion of the thickness, it refers to the thickness at the position where the pair of score markings is hollowed.

According to the invention, the product can be divided into two parts by, for example, exerting traction on each side of the at least one score marking. If other score markings are present, it is also possible to divide the product into smaller parts by pulling again along these score markings. As many parts as desired, for example perfectly identical, are thus obtained.

The force applied to divide the product according to the invention can be a tensile force (one pulls on each of the sub-parts of the product by moving them away from each other or one pulls on a single part in the direction of the length of the score marking) or a force of pressure (one of the parts is pressed with the hand or the finger while the other part is maintained in the other hand).

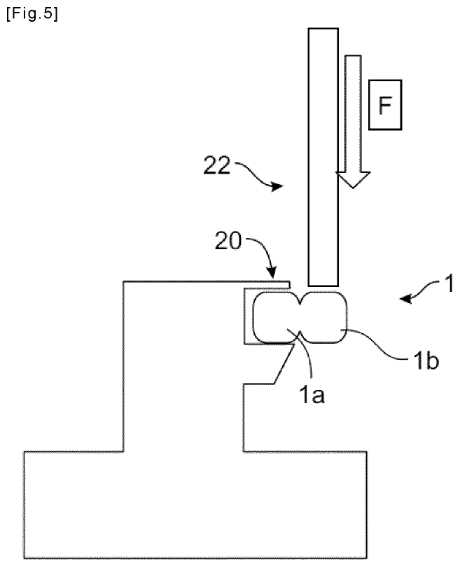

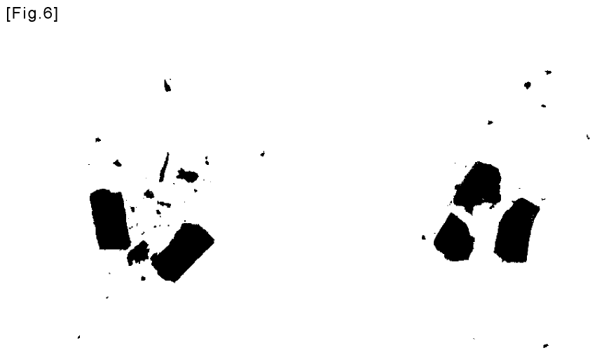

As illustrated in FIG. 5, within the meaning of the invention, the force required to divide the product by 10 to 60 N, along the at least one score marking, is understood as the force measured on a texturometer, in particular TA XT2i (Stable Micro Systems) at a temperature between 18 and 25° C. using a 2 cm diameter cylindrical probe with the following parameters:

### Compression in Test Mode

Pre-test speed 1.00 mm/sec
Test speed 10.00 mm/sec
Post-test speed 10.00 mm/sec
Target mode distance
Distance 5.00 mm
Trigger Type Auto (Force)
Release force 0.5 N

These parameters are, for example, implemented directly in the texturometer software.

Briefly, a first half of the product was fixed in a jaw 20 and a cylindrical probe 22 was lowered just above the product 1 serving as a sample (1 mm) and then lowered at a distance of 5 mm at a predetermined speed of 10 mm/sec.

The cylindrical probe 22 exerts pressure on a second half of the product until division. The product is placed in the jaw 20 so that the division of the sample resulting from the test takes place along the at least one score marking 5, 6. The resistance offered by the sample (force necessary to divide the sample) has been recorded. The measured resistance expressed in N corresponds to the division force.

Advantageously, the score markings on the product, is configured so that the force necessary to divide the product into doses is between 20 and 55 N, or even between 24 and 52 N.

Advantageously, the composition of the product and/or of the score markings of the product, are configured so that the loss of mass during the division is less, in particular strictly less, than 0.4%, preferably 0.3%, more preferably 0.2% of the total mass of the product, even more preferably less than 0.1% of the total mass of the product.

In order to minimize the loss of mass during division, the composition of the product according to the invention provides a friability, as determined according to the method of the European Pharmacopoeia, chapter 2.9.7 (Friability of uncoated tablets) of less than 0.5% and preferably less than 0.2%.

It is possible to provide score markings having a rounded, square or rectangular section, for example, so that the width of said score marking is constant over their entire depth. Score marking having a U or V section are particularly suitable.

When the score marking has a V-section, as in FIGS. 1 to 3, the V-section has an opening angle of between 40° and 120°, advantageously between 6° and 100° and preferably equal to 80°+/−5°. The V-section is understood along a section plane perpendicular to a large dimension of the product.

According to a first aspect, not shown, the product has a score marking provided on a single surface of the product, preferably in the thickness of the product. In this configuration, the score marking has a depth greater than 20%, advantageously greater than 25%, and preferably greater than 50%, of a thickness of the product. The cumulative depth of the score markings is advantageously less than 90% of the thickness of the product. Beyond such a depth, the product is then too fragile and risks separating into several doses, without intentional division during storage or when the product is taken out of its packaging.

According to a preferred aspect of the invention, as illustrated in FIGS. 1 to 3, the product has at least one pair of score markings 5, 6 facing each other on opposite faces 3, 4 of the product. In this configuration, the cumulative depth of the two score markings is preferably greater than 20%, more preferably greater than 30%, even more preferably greater than 40% and in a specific embodiment greater than 50%. The cumulative depth of the score markings is less than 90% of a product thickness. Beyond such a depth, the product is then too fragile and risks separating into several doses, without intentional division during storage or when the product is taken out of its packaging. Advantageously, the cumulative depth of the score markings 5, 6 or of the pair of score markings is less than 70% of a thickness of the product.

In this configuration, the score markings 5, 6 facing each other may have the same depth or a different depth from each other.

According to one embodiment, each score marking has a different depth from the opposite score marking, said depth being between 5% and 80%, the cumulative depth of the two score markings 5, 6 being greater than 10%, greater than 20%, more preferably greater than 30%, even more preferably greater than 40% and in a specific embodiment greater than 50% and less than 90%, advantageously less than 70%.

According to another advantageous embodiment, corresponding to the illustrated embodiments, the score markings 5, 6 facing each other have an identical depth, for example between 5% and 45%, advantageously between 10 and 35%, preferably between 25 and 35% of the thickness of the product, the cumulative depth of the two score markings being greater than 10%, greater than 20%, more preferably greater than 30%, even more preferably greater than 40% and in a specific embodiment greater than 50%, and less than 90%, advantageously less than 70%.

When the product according to the invention is intended to be divided into two parts, the product has a single score markings hollowed out on a single surface of the product or, as in FIG. 1, a single pair of score markings 5, 6 located opposite each other. -screw on the opposite faces 3, 4 of the product.

The score marking or score markings facing each others can be cut into the thickness of the product so as to delimit two identical parts, as in FIG. 1. Alternatively, they may be two different parts.

The choice of the configuration of the score markings facing each other will depend on the desired division and on the dose of substance, in particular of active substance, to be administered. For an active substance to be administered in two identical doses, the score marking or score markings 5, 6 facing each other will be configured so as to delimit two identical parts of the product. For an active substance to be administered in a first dose representing ⅓ of the total quantity of active substance, and a second dose representing ⅔ of the total quantity of active substance, the score marking or score markings facing each other will be configured so as to delimit two parts representing respectively ⅓ and ⅔ of the product.

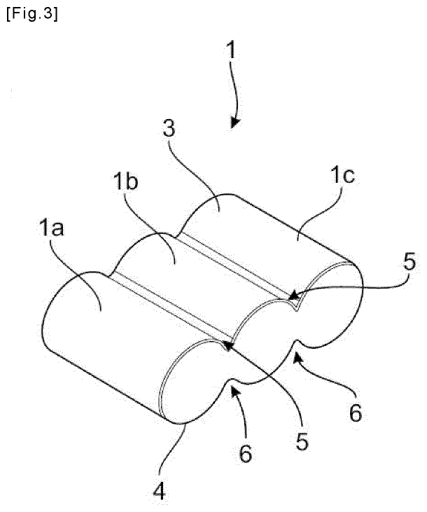

As illustrated in FIGS. 2 and 3, when the product according to the invention is intended to be divided into more than two parts, the product has, in addition to the first score marking or the pair of score markings facing each other, at least one other score marking hollowed out on a single surface of the product or, as in FIGS. 2 and 3, another pair of score markings located facing each other on opposite sides of the product, said score marking or pairs of score markings being configured to allow division of the product into a plurality of doses.

The pairs of score markings can be configured to allow a division of the product into as many parts or doses as necessary.

As illustrated in the figures, the pairs of score markings are cut on the same surface or surfaces of the product.

The pairs of score markings can be dug in a parallel direction, as in FIG. 3 and/or in transverse directions, as in FIG. 2.

The pairs of score markings can delimit identical doses such as thirds of product as in FIG. 3 or quarters of product as in FIG. 2 or even different doses such as a half product and two quarters of product.

According to a first variant, the depth of the score markings is identical, whether they are the score markings of the same pair and/or all the score markings.

As a variant, it is also possible to provide additional score markings or pairs of score markings of different depths from the first score marking or first pair of score markings in order to favor the division of the product according to one of the score markings. For example, a first deeper score marking or pairs of score markings may be provided to allow the division of the product into two halves and other score markings or pairs of score markings of lesser depth for the division of each half into smaller parts.

The three-dimensional form of the product according to the invention can be very diverse. It can be a composition of superimposed cylinders, triangles, etc. Preferably, the product has an elongated cylindrical, elliptical, oblong, parallelepiped or cuboid form.

In FIG. 1, it has the form of two parallel cylinders, each cylinder corresponding to one of the doses 1a, 1b and being connected to the other cylinder by the pair of score markings 5, 6. In FIG. 2, it has a substantially parallelepiped form with small rounded sides. It forms four cuboid doses connected by pairs of orthogonal 5, 6 score markings. In FIG. 3, it has the form of three parallel cylinders, each cylinder corresponding to one of the doses 1a, 1b, 1c and being connected to another of the cylinders by one of the pairs of score markings 5, 6.

The products according to the invention advantageously have a mass of between 100 mg and 20 g, in particular between approximately 100 mg and approximately 10 g, even more particularly between 500 mg and 7 g. In illustrative embodiments, the products have a mass of 700 mg, 3.5 g and 6.5 g.

The products according to the invention are intended to be administered to all types of subjects, human and non-human. Advantageously, the products according to the invention are intended for non-human mammals.

The formed soft-chewable products according to the invention can thus be formulated in the form of appetizing bites or soft-chewable tablets.

The formed soft-chewable products according to the invention are particularly suitable for administration to companion animals, in particular dogs, cats and ferrets.

According to an advantageous embodiment, the formed soft-chewable products are intended for subjects having difficulty in ingesting large drugs, such as geriatric or pediatric subjects.

The products according to the invention can be used for the administration of all types of substances, in particular pharmaceutical or veterinary active substances or nutraceutical substances.

The products of the present invention therefore comprise at least one nutraceutical substance or at least one active pharmaceutical substance, distributed homogeneously within the product.

In one embodiment, the active substance is a pharmaceutical active ingredient, in particular chosen from anesthetics, corticosteroids, NSAIDs, antibiotics, antiemetics, antithyroid agents, anthelmintics, parasiticides, appetite stimulants, antihistamines, antipruritics, histamine blockers, antifungal agents, antiprotozoal agents, anthelmintics, antidepressants or steroids, or any combination of two or more of these.

The NSAID can be selected from meloxicam, carprofen, naproxen, tolfenamic and meclofenamic acids, flunixin and indomethacin.

The anthelmintic can be chosen from macrocyclic lactones, such as abamectin, moxidectin, avermectins, ivermectin, selamectin or cydectin, milbemycin; benzimidazoles such as mebendazole, fenbendazole, oxfendazole, albendazole, thiabendazole or carbendazole; imidazothiazoles such as levamisole or pyrantel; salicylanilides such as closantel or niclosamide; praziquantel or epsipantel.

The antibiotic can be chosen from penicillins, aminopenicillins (for example, amoxicillin, ampicillin, hetacillin), cephalosporins (for example, cefovecin, cefuroxime, ceftiofur), quinolones (for example, ciprofloxacin, enrofloxacin, difloxacin, orbifloxacin, marbofloxacin); chloramphenicols (eg, chloramphenicol, thiamphenicol, florfenicol); tetracyclines (eg, chlortetracycline, tetracycline, oxytetracycline, doxycycline, minocycline); macrolides (eg, erythromycin, tylosin, tlimicosin, clarithromycin, azithromycin, tulathromycin); lincosamides (eg, lincomycin, clindamycin); aminoglycosides (eg, gentamicin, amikacin, kanamycin, apramycin, tobramycin, neomycin, dihydrostreptomycin, paromomycin).

The antiemetic can be chosen from maropitant and aprepitant.

The parasiticidal agent can be chosen from organochlorines, organophosphates, carbamates, amidines, pyrethrins and synthetic pyrethroids, benzoylureas, analogues of juvenile hormones, macrocyclic lactones, neonicotinoids, phenylpyrazoles, spinosyns, such as spinosad, isoxazolines, such as fluralaner, afoxolaner, sarolaner and lotilaner.

The antipruritic is a JAK-1 inhibitor, selected in particular from oclacitinib, momelotinib, tofacitinib and ruxolitinib.

Within the meaning of the present invention, a nutraceutical ingredient refers to an ingredient whose purpose is to help or maintain the health of the target animal. For example, the nutraceutical ingredient may provide a nutritional benefit to the target animal. Nutraceutical ingredients include, but are not limited to, vitamins, minerals, plant extracts and combinations thereof.

Vitamins include, for example, vitamin A, vitamin E, vitamin B12, vitamin B3, d-pantothenic acid (vitamin B5), folic acid, vitamin B6, vitamin B1, vitamin D3, vitamin C, vitamin B2 and provitamins such as beta-carotene or panthenol.

Minerals include potassium, sodium, manganese, zinc, iron, calcium, copper, cobalt, iodine, chlorine and selenium. The mineral may be in the form of a suitable salt.

Other nutraceutical ingredients being considered include lecithins, phosphatidylserine, antioxidants such as Pycnogenol, Gingko biloba extracts, Bacopa monnieri extracts, huperzine A, omega-3 polyunsaturated fatty acids such as DHA, I EPA; flavonoids or plant extracts containing flavonoids such as blueberry or maqui berry, anthocyanins, flavan-3-ols, epigallocatechin-gallate (ECGC) and proanthocyanidins, isoflavones, eg puerarin and genistein and flavonols, for example rutin and troxerutin; cytochrome C, monosodium glutamate, N-acetyl cysteine, zinc methionine, biotin, L-carnitine, phytoceramides, prebiotics, probiotics, postbiotics, casein phosphopeptides, L-theanine or green tea extract, ashwaganda and fish protein hydrolysates.

In a particular embodiment, the nutraceutical ingredient is selected from the group consisting of eggshell membrane; krill powder or krill oil, astaxanthin or a source of astaxanthin such as seaweed, yeast, salmon, trout, krill, shrimp and crayfish; Boswellia serrata; hyaluronic acid or its salts; and vitamin D3. Preferably, the nutraceutical is a mixture of at least 2, at least 3 or at least 4 nutraceutical ingredients selected from the group consisting of eggshell membrane; krill powder, astaxanthin or a source of astaxanthin such as seaweed, yeast, salmon, trout, krill powder or krill oil, shrimp and crayfish; Boswellia serrata; hyaluronic acid or its salts; and vitamin D3. Even more preferentially, the nutraceutical is a mixture of eggshell membrane, krill powder or krill oil, astaxanthin, Boswellia serrata and hyaluronic acid or its salts.

The pharmaceutical, veterinary or nutraceutical active substance may be present in the product in any amount as needed to achieve the desired therapeutic effect or health benefit. Advantageously, the pharmaceutical, veterinary or nutraceutical active substance is present at a level of 0.1 to 20%, preferably at a level of 1 to 10% of the total mass of the product.

Of particular note is a soft-chewable product according to the present invention wherein the active is meloxicam.

The invention also proposes a process for manufacturing a formed soft-chewable product for oral administration, as described above.

Said method comprises, for example, the steps of:
(a) obtaining a composition comprising a pharmaceutical, veterinary or nutraceutical active substance and inactive ingredients such as excipients,
(b) forming the composition to obtain said product.

The products according to the invention can advantageously be manufactured by extrusion, by molding or by compression of the composition.

The present invention therefore relates to a process for manufacturing a formed soft-chewable product for oral administration, comprising a pharmaceutical, veterinary or nutraceutical active substance, a humectant and a forming agent, wherein the product presents a pair of score markings facing each other on opposite faces of the product and configured for a division of the product into doses, the cumulative depth of the pair of score markings being greater than 10% of a thickness of the product and the cumulative depth of the two score markings being less than 90% of a thickness of the product, and the loss of mass during the division of the product (1) into doses (1a, 1b) being less than 0.5%.

In more detail, said method comprises, for example the steps of:
a) mixing a pharmaceutical, veterinary or nutraceutical active substance with a humectant and a forming agent and optionally inactive ingredients such as excipients to prepare said composition,
c) molding, compressing or extruding the composition under conditions, in particular temperature and pressure, suitable to obtain a soft-chewable product, the pair of score markings being hollowed into the product by the mold during compression or molding or the die during extrusion.

In one embodiment, the invention relates to a process for manufacturing a product as defined above, in which the product is formed by extrusion of the composition, the at least one score marking 5, 6 being hollowed in the product by the die of the extruder.

According to this embodiment, the pharmaceutical, veterinary or nutraceutical active substance and the inactive ingredients such as the excipients are introduced into a hopper of the extruder, advantageously after the mixing step. The composition is then pushed through the extruder under suitable conditions to give the extrudate the desired structural characteristics, such as a firmness between 10 and 300 N and friability of less than 1%, until it leaves the extruder. At the exit of the extruder, a die of suitable shape forms the pair of score markings 5, 6 in the extrudate.

The extrudate, in which at least one score marking is hollowed out, is cut in a direction transverse to the length of the extrudate into portions having the desired length, each portion thus obtained representing a product according to the invention.

It is preferable for the pair of score markings to extend over a greater dimension of the product because this avoids a too high cutting frequency of the extrudate, thereby making it possible to obtain better mass uniformity between the unitary products.

The present invention thus relates to a method for preparing a formed soft-chewable product by extrusion, for oral administration as described above, comprising the steps of:
(a) optionally, obtaining the composition formed of the pharmaceutical, veterinary or nutraceutical active substance, a humectant and a forming agent, and optionally inactive ingredients such as excipients in a mixer,
(b) introducing the active pharmaceutical, veterinary or nutraceutical substance, a humectant and a forming agent and inactive ingredients such as excipients into the hopper of the extruder to obtain said composition or introducing of said composition previously formed in step (a) into the hopper of the extruder,
(c) extruding said composition under suitable conditions to give an extrudate,
(d) hollowing the pair of score markings by the die of the extruder,
(e) cutting the extrudate to obtain the formed soft-chewable product for oral administration,
(f) optionally, packaging the product.

When the product has several score markings or several pairs of score markings in a parallel direction, these can be formed on the product directly using a die of appropriate shape. A die will then be used in which the number of score markings or pairs of score markings desired on the product is made.

When the product has at least one score markings or pair of score markings, called additional score markings, in a direction transverse to the other score marking(s) or pair of score markings, called first score markings, an additional step is then necessary to form the said additional score marking(s) or pair of additional score markings.

Said additional score markings or pairs of additional score markings can be formed on the product by any means. According to one embodiment, the additional score markings or pairs of score markings are formed on the product by molding or by compression.

In this embodiment, the product obtained by extrusion is introduced into an apparatus such as a tablet press, a forming machine of the Formax F6™ type manufactured by Formax Corporation, the forming machines described in U.S. Pat. Nos. 3,486,186; 3,887,964; 3,952,478; 4,054,967; 4,097,961; 4,182,003; 4,334,339; 4,338,702; 4,343,068; 4,356,595; 4,372,008; 4,535,505; 4,597,135; 4,608,731; 4,622,717; 4,697,308; 4,768,941; 4,780,931; 4,818,446; 4,821,376; 4,872,241; 4,975,039; 4,996,743; 5,021,025; 5,022,888; 5,655,436; and 5,980,228, or the machine described in patent EP2922526 and the product is then compressed or molded to form the additional score marking or pair of score markings.

The present invention thus relates to a method for preparing a formed soft-chewable product by extrusion, for oral administration as described above, comprising the steps of:
(a) optionally, obtaining the composition formed of the pharmaceutical, veterinary or nutraceutical active substance, a humectant and a forming agent and optionally the inactive ingredients such as excipients in a mixer,
(b) introducing the active pharmaceutical, veterinary or nutraceutical substance, a humectant and a forming agent and optionally inactive ingredients such as excipients into the hopper of the extruder to obtain the composition or introducing said composition previously formed in step (a) into the hopper of the extruder,
(c) extruding said composition, and hollowing the pair of score markings through the die of the extruder,
(d) cutting the extrudate,
(e) forming an additional score marking or pair of score markings on the product, preferably by compression or by moulding, under conditions suitable to give a soft-chewable product,
(f) optionally, product packaging.

In another embodiment, the invention relates to a process for manufacturing a product as defined above, in which the score marking is formed in the product by molding or compression, without prior extrusion.

According to this embodiment, a mixture comprising the active substance and the excipients of the composition are introduced into a mixer then the composition (of paste type) obtained is subjected to a step of compression or molding in an apparatus such as a press tablets, a forming machine of the Formax F6™ type manufactured by Formax Corporation, the forming machines described in U.S. Pat. Nos. 3,486,186; 3,887,964; 3,952,478; 4,054,967; 4,097,961; 4,182,003; 4,334,339; 4,338,702; 4,343,068; 4,356,595; 4,372,008; 4,535,505; 4,597,135; 4,608,731; 4,622,717; 4,697,308; 4,768,941; 4,780,931; 4,818,446; 4,821,376; 4,872,241; 4,975,039; 4,996,743; 5,021,025; 5,022,888; 5,655,436; and 5,980,228, or the machine described in patent EP2922526, the score marking or pairs of score markings being formed using a mold of appropriate shape.

This method makes it possible, by using an appropriate mould or punch, to manufacture a product having a pair of score markings as well as an additional score marking or pair of additional score markings, whatever the direction of the additional score markings, in particular in a direction parallel to the first score marking or pair of score markings and/or in a direction transverse to the first score marking or pair of score markings, in a single step.

The present invention thus relates to a process for the preparation of a soft-chewable product shaped for oral administration as described above comprising the steps of:
(a) optionally, obtaining the composition formed of the pharmaceutical, veterinary or nutraceutical active substance, humectant and forming agent and optionally the inactive ingredients such as excipients in a mixer,
(b) introducing the pharmaceutical, veterinary or nutraceutical active substance, humectant and forming agent and optionally inactive ingredients (excipients) to obtain the composition, or introducing the composition obtained in step (a) into a container connected to a compression or molding machine,
(c) compressing or molding the composition under suitable conditions to give a soft-chewable product, the pair of score markings being hollowed in the product by the mould,
(d) optionally, packaging the product.

According to this embodiment, it is preferable to mix in a mixer the pharmaceutical, veterinary or nutraceutical active substance, a humectant and a forming agent and optionally the inactive ingredients (excipients) before proceeding to the compression or molding step, so as to guarantee a uniform distribution of the pharmaceutical, veterinary or nutraceutical active substance in the composition.

The present invention also particularly relates to a soft-chewable product for oral administration, as described above, for its use as a medicament.

According to an advantageous aspect of the invention, the product is intended to be administered in the form of doses concomitantly to several subjects and/or to be administered in the form of sequential doses to the same subject.

Most pharmaceutical, veterinary or nutraceutical active substances are administered according to a dosage established according to the weight of the subject. Since the product according to the invention can be divided into several parts, it is thus possible to precisely adjust the quantity of active pharmaceutical substance to the weight of the subject to be treated.

A “dose” within the meaning of the present invention represents the portion of the product making it possible to obtain the desired therapeutic effect in the subject to be treated. The term “portion” and the term “dose” are therefore interchangeable in the present application.

The dose of effective pharmaceutical, veterinary or nutraceutical active substance corresponds to the quantity recommended by the practitioner, which is generally known in the literature and/or determined by the person skilled in the art on the basis of his general knowledge or by carrying out preliminary or clinical tests.

Advantageously, the present invention relates to a product as defined above, containing an active substance, in particular veterinary, in particular intended for a pet such as a dog, a cat or a ferret, for its use as a medicament as described below.

More generally, the present invention relates to a soft-chewable product for oral administration as described above, having a pair of score markings configured to promote division of the product into doses, the strength necessary to divide the product into doses being preferably between 10 and 60 N and the loss of mass during the division of the product into doses being less than 0.5%, comprising a pharmaceutical, veterinary or nutraceutical active substance, for its use as a medicament.

Advantageously, a dose of the product is intended to provide the subject to be treated with the effective amount of pharmaceutical, veterinary or nutraceutical active substance until the next administration of the pharmaceutical, veterinary or nutraceutical active substance to be administered to the subject.

The possibility of adjusting the quantity of product to the weight of the subject to be treated reduces the risk of overdose. In addition, since the division of the product takes place without loss of mass, the quantity of product administered is precise and no under-dosing results from the division of the product. For example, a precise quantity of the active substance can be administered to subjects whose weight varies widely with the same product. For example, if each portion of the product contains the effective quantity of active substance for 10 kg of the subject's weight, a 30 kg subject will receive ⅜ of the product and a 70 kg subject will receive ⅞ of the product. This avoids overdosing or under-dosing the subject.

According to one aspect of the invention, the product is intended to be administered in several doses at defined intervals, each dose providing the subject to be treated with the quantity of active pharmaceutical, veterinary or nutraceutical substance effective until the administration of the following dose.

The same product can be administered in several successive doses when the dosage requires administration in several times. For example, when the product is to be administered twice a day, the product can be cut in half and each dose taken at the indicated time of the day.

The same product can also be administered in several successive doses when the subject has difficulty in swallowing a whole product, for example when the subject is a geriatric subject or a pediatric subject. As the product can be divided into smaller and therefore easy to swallow doses, it helps to ensure that the treatment is taken in its entirety, without under-dosing due to the division of the product.

Thus, said product is optionally intended to be administered in several doses for a single treatment to a geriatric subject or to a pediatric subject.

Preferably, the product is intended for a companion animal, in particular a dog or a cat. In one embodiment, the product comprises a nutraceutical active substance.

The present invention particularly concerns a soft-chewable product as described above, for use as a medicament, wherein an effective dose of the product is administered sequentially to an animal in need thereof.

In a particular embodiment, the present invention relates to a soft-chewable product as described above for use as a medicament, wherein the product is divided into doses, and each dose is administered at defined intervals.

By “defined intervals”, it is meant periods of time corresponding to the posology of the drug. For example, said interval may be some hours, e.g. 4 hours, 6 hours, 12 hours or 24 hours, some days, e.g. 2 days, 3 days, 7 days or some weeks, e.g. 2 weeks, 3 weeks or 4 weeks.

Preferably, the product is divided in two equivalent doses, the second dose being administered within 24 hours of the administration of the first dose. In one particular embodiment, the present invention concerns a soft-chewable product as described above for use as a medicament, wherein the product is administered once a day in two doses.

The present invention also relates to a method of treating a pathology or a condition in a patient, comprising the following steps:
(1) providing a soft-chewable product 1 comprising a pharmaceutical, veterinary, or nutraceutical active substance, a humectant, and a forming agent, wherein said product 1 presents a pair of score markings 5, 6 facing each other on opposite faces 3a, 3b of the product,
(2) dividing the soft-chewable product 1 into individual doses along the score markings, said doses being adapted to the patient, in particular its weight,
(3) repeating the administration of the individual doses at defined intervals based on the recommended therapeutic regimen for the pathology.

The present invention also relates to a method for treating a subject against a pathology, the method comprising the administration to said subject of a dose of a soft-chewable product for oral administration comprising a pharmaceutical, veterinary or nutraceutical active substance, a humectant and a forming agent, said product having a pair of score markings configured to favor a division of the product into doses, the force necessary to divide the product into doses being between 10 and 60 N and the loss of mass when dividing the product into doses being less than 0.5%, said dose providing the subject to be treated with the amount of effective pharmaceutical, veterinary or nutraceutical active substance until the next administration of said pharmaceutical, veterinary or nutraceutical active substance.

The present invention further relates to a method for treating a subject against a pathology, the method comprising the sequential administration to said subject of several doses of a soft-chewable product for oral administration comprising a pharmaceutical, veterinary or nutraceutical active substance, a humectant and a forming agent, said product having a pair of score markings configured to favor division of the product into doses, the force necessary to divide the product into doses being between 10 and 60 N and the loss of mass during the division of the product in doses being less than 0.5%, providing the subject to be treated at each administration with the effective dose of the pharmaceutical, veterinary or nutraceutical active substance until the administration of the next dose.

The present invention further relates to the use of a soft-chewable product for oral administration comprising a pharmaceutical, veterinary or nutraceutical active substance, a humectant and a forming agent, said product having a pair of score markings configured to favor division of the product into doses, the force necessary to divide the product into doses being between 10 and 60 N and the loss of mass during the division of the product in doses being less than 0.5%, for the manufacture of a medicament intended for treating a subject against a pathology.

The present invention further relates to the use of a soft-chewable product for oral administration comprising a pharmaceutical, veterinary or nutraceutical active substance, a humectant and a forming agent, said product having a pair of score markings configured to favor division of the product into doses, the force necessary to divide the product into doses being between 10 and 60 N and the loss of mass during the division of the product in doses being less than 0.5%, for the manufacture of a medicament intended for treating a subject 20 against a pathology, the product being administered to the subject in several doses.

The present invention further relates to the use of a soft-chewable product for oral administration comprising a pharmaceutical, veterinary or nutraceutical active substance, a humectant and a forming agent, said product having a pair of score markings configured to favor division of the product into doses, the force necessary to divide the product into doses being between 10 and 60 N and the loss of mass during the division of the product in doses being less than 0.5%, for the manufacture of a medicament intended for treating a subject against a pathology, the product being administered to the subject in several doses and providing each day the quantity of effective pharmaceutical, veterinary or nutraceutical active substance until the administration of the next dose.

The present invention further pertains to a treatment schedule, wherein the soft-chewable product as described above is administered in equal doses at regular intervals.

The present invention is in particular directed to a soft-chewable product 1 for oral administration, comprising a pharmaceutical, veterinary or nutraceutical active substance, a humectant and a forming agent, said product 1 comprising a pair of score markings 5, 6 facing each other on opposite faces 3a, 3b of the product and configured for a division of the product into doses 1a, 1b, the force necessary to divide the product 1 into doses 1a, 1b along the pair of score markings 5, 6 being preferably between 10 and 60 N, and the loss of mass during the division of the product 1 into doses (1a, 1b) being less than 0.5%.

Preferably, in said product 1, the cumulative depth of the two score markings 5, 6 is greater than 20%, preferably greater than 30%, even more preferably greater than 40%, and in a specific embodiment greater than 50%, of a thickness of the product 1 and the cumulative depth of the two score markings 5, 6 is less than 90%, advantageously less than 70%, of a thickness of the product 1. More preferably, the two score markings 5, 6 have an identical depth, each of the score markings 5, 6 having a depth of between 10% and 45%, advantageously between 25% and 35%, of a thickness of the product 1.

In one embodiment, the score markings (5, 6) comprise cutting primers.

Preferably, the product has a friability of less than 0.5%, and more preferably less than 0.2%.

Preferably, the product comprises:
a pharmaceutical, veterinary or nutraceutical active substance,
a humectant selected from the group consisting in propylene glycol, glyceryl triacetate, vinyl alcohol, neoagarobiose, sugar polyols such as glycerol, sorbitol, xylitol and maltitol, polymeric polyols such as polydextrose, caprylic/capric triglyceride, propylene glycol dicaprilate/dicarprate, medium chain triglycerides sold under the trademark Miglyol, especially Miglyol 812 or 814, vegetable oils, or mixtures thereof, and
a forming agent selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline cetyl wax, alcohol, polysaccharides, hydroxypropylmethylcellulose, poly(meth)acrylates, alkyl poly(meth)acrylates, and mixtures thereof.

Preferably, the product further comprises:
a filler, preferably selected from the group consisting in starch such as corn starch, rice starch or potato starch, sucrose, confectioner's sugar, lactose including anhydrous lactose, hydrated lactose, sprayed dried lactose, dextrin, dextrate, mannitol, soy grits, soy protein fines microcrystalline cellulose, silicified microcrystalline cellulose, calcium phosphate, crystalline maltose, maltodextrins and mixtures thereof,
a palatant, preferably selected from the group consisting in animal product-based palatants such as dried or powdered meat and meat parts such as beef, pork, chicken, turkey, fish, and lamb; organ meats such as liver and kidney; meat meals, bone meals and ground bone and non-animal product based palatants such as essential oil, oleoresin, essence or extract, protein hydrolysate, distillate, or any product of roasting, heating or enzymolysis, which contains the flavoring constituents derived from a spice, fruit or fruit juice, vegetable or vegetable juice, edible yeast (active and inactive), herb, bark, bud, root, leaf or any other edible portions of a plant, and mixtures thereof.

Preferably, the product further comprises excipients.

Preferably, the product is for its use as a medicament, for human or veterinary applications.

Preferably, the product is intended for a geriatric subject or a pediatric subject.

Preferably, the product is intended for a dog or a cat, preferably a dog.

Preferably, the product is manufactured by extrusion.

Preferably, the product is prepared by the following process:
(a) optionally, obtaining the composition formed of the pharmaceutical, veterinary or nutraceutical active substance and the inactive ingredients such as excipients in a mixer,
(b) introducing the active pharmaceutical, veterinary or nutraceutical substance and inactive ingredients such as excipients into the hopper of the extruder to obtain said composition or introducing of said composition previously formed in step (a) into the hopper of the extruder,
(c) extruding said composition under suitable conditions to give an extrudate,
(d) hollowing the pair of score markings by the die of the extruder,
(e) cutting the extrudate to obtain the formed soft-chewable product for oral administration,
(f) optionally, packaging the product.

### EXAMPLES—CHARACTERIZATION OF THE PRODUCTS ACCORDING TO THE INVENTION

The product tested has the following composition (percentages by mass relative to the total mass):

About 3-5% active ingredient; 4-6% microcrystalline cellulose; 4-6% icing sugar; 4-6% polyethylene glycol 3350; 15-25% native corn starch; 5-15% croscarmellose sodium; 25-35% palatant; 0.1-5% sodium chloride; 1-10% purified water; 5-20% glycerin; 1-10% soybean oil; 0.001-1% antioxidants and preservatives.

### Example 1.1—Firmness of Products

The firmness of the product is determined according to the method described above.

The average value measured is 65 N.

### Example 1.2—Friability

The friability of the products was measured according to the method of the European Pharmacopoeia, chapter 2.9.7 (Friability of uncoated tablets) using 12 divisible product halves. The results are reported in Table 1. The friability is represented by the mass loss in %.

TABLE 1

| Mass of the product | 700 mg | 3.50 g | 6.50 g |
|---|---|---|---|
| Mass before the test (mg) | 4.331 | 20.856 | 38.541 |
| Mass after the test (mg) | 4.326 | 20.839 | 38.523 |
| Mass loss in % | 0.12 | 0.08 | 0.05 |

### Example 1.3—Secability: Tensile Force and Loss of Mass

The tensile force was measured according to the method explained above.

The test was performed on samples of approximately 700 mg, approximately 3.5 g and approximately 6.5 g (6 of each) after each product was weighed. The results are given in Table 2:

TABLE 2

| | 700 mg | 3.5 g | 6.5 g |
|---|---|---|---|
| min strength | 21.96 | 28.88 | 44.26 |
| max strength | 27.37 | 39.98 | 61.98 |
| average strength | 24.13 | 36.25 | 51.81 |
| standard deviation | 1.97 | 4.59 | 6.77 |

After the division test, the product halves were weighed again to assess the loss of mass following the division. The results are given in Table 3:

TABLE 3

| | | 700 mg | 3.5 g | 6.5 g |
|---|---|---|---|---|
| Mass before division | 1 | 709 | 3.419 | 6.490 |
| | 2 | 733 | 3.477 | 6.448 |
| | 3 | 728 | 3.514 | 6.422 |
| | 4 | 725 | 3.647 | 6.26 |
| | 5 | 752 | 3.362 | 6.470 |
| | 6 | 686 | 3.44 | 6.463 |
| Masse after division | 1 | 708 | 3.419 | 6.490 |
| test (2 halves) | 2 | 733 | 3.474 | 6.448 |
| | 3 | 728 | 3.509 | 6.422 |
| | 4 | 725 | 3.647 | 6.259 |
| | 5 | 752 | 3.361 | 6.470 |
| | 6 | 686 | 3.44 | 6.463 |
| Mass loss (%) | 1 | 0.1 | 0.0 | 0.0 |
| | 2 | 0.0 | 0.1 | 0.0 |
| | 3 | 0.0 | 0.1 | 0.0 |
| | 4 | 0.0 | 0.0 | 0.0 |
| | 5 | 0.0 | 0.0 | 0.0 |
| | 6 | 0.0 | 0.0 | 0.0 |

It can be seen that each of the samples tested has the values specified for the firmness of the product in accordance with the invention. In addition, the presence of at least one score marking associated with the force necessary to divide the product along the score marking or score markings, according to the values specified for the product in accordance with the invention, allows a division of the product with an extremely low mass loss.

### Example 1.4—Comparison Between Soft Chews with One Score-Marking and Soft Chews Having a Pair of Score Markings Facing Each Other on Opposite Faces (3a, 3b) of the Product

The products were either soft chews with a single score-marking on one face or a pair of score markings facing each other on opposite faces of the product. The score-marking or score markings had an identical depth having a cumulative depth of 20% of the thickness of the soft-chew. The formula was the same for both types of soft-chews.

The objective of this example was to evaluate the effect of the introduction of a second score marking facing the first one on the opposite face of the product.

Two parameters were evaluated: the mass loss and the precision of the division. For that, 30 soft chews of each type (one score marking or two score markings) were individually weighted. Then, these 30 soft-chews were divided along the score-marking(s) by hand by three different users (10 soft-chews by user). Each halve of the soft-chew was weighted and used to make a statistical evaluation.

For both type of soft chews, the mass loss was not significantly different and in both cases less than 0.01% based on the total weight of the soft-chew.

There were significant differences between the weights of the halves of the divided soft-chews. The results are presented in the next table:

| | One score- | Pair of score- |
|---|---|---|
| | marking | markings |
| Average weight of the whole soft-chew | 3.38 ± 0.04 | 3.32 ± 0.05 |
| Average weight of the Heaviest halve | 1.78 ± 0.06 | 1.69 ± 0.02 |
| Average weight of the lighter halve | 1.59 ± 0.04 | 1.63 ± 0.04 |

It can be seen that the mass difference between the weights of the halves is higher for the soft-chews with one score marking that for the soft-chews with a pair of score markings.

Thus, the presence of a pair of score markings facilitates the division of the soft-chews in two identical doses.

The mass difference in percent between the doses is also statistically different: the dose difference between the doses of a soft-chew are 2±1.5% when two score markings are present and 5.9±3.5% when only one score marking is present. This indicates that the risk of overdosing or under-dosing is higher when only one score marking is present on the soft-chew.

### Example 1.5—Comparison Between Soft Chews without a Pair of Score Markings Facing Each Other on Opposite Faces of the Product Differing in their Friability

The products were soft chews of about 3.5 g without score markings differing in their friability, having a firmness of about 60 N).

The objective of this example was to evaluate the effect of the friability of the soft-chew on the ease and precision of the division in real-life conditions. For that, 20 soft chews of each type were individually weighted. Then, these 20 soft-chews were divided by hand by two different users (10 soft-chews by user). During this test, it was rapidly seen that the soft chews with a high friability, despite being acceptable to be used as a whole, were not acceptable to be used in doses.

Indeed, in almost all cases, the soft-chews crumbled in the hands of the user due to the pressure exerted on the product by the fingers or crumbled along the score markings when tearing the two doses apart, resulting in an unacceptable mass loss and a division in uneven doses. [FIG. 6] shows soft-chews having a high friability after an attempt to divide them by hand in two parts. As can be seen on the figure, the soft-chews crumble upon division and several pieces are observed. The mass loss is hence unacceptable. In one case (right picture), the soft-chew broke apart even in three major pieces and other small pieces also broke apart.

Thus, a low friability of the soft chews helps the clean and even division of the soft-chew.

1. A soft-chewable product (1) for oral administration, comprising a pharmaceutical, veterinary or nutraceutical active substance, a humectant and a forming agent,
wherein the product (1) presents at least a pair of score markings (5, 6) facing each other on opposite faces (3a, 3b) of the product and configured for a division of the product into doses (1a, 1b), the cumulative depth of the pair of score markings (5, 6) being greater than 10% of a thickness of the product (1), and the loss of mass during the division of the product (1) into doses (1a, 1b) is less than 0.5%.

2. The soft-chewable product (1) according to claim 1, wherein the cumulative depth of the two score markings (5, 6) is greater than 20% of a thickness of the product (1) and the cumulative depth of the two score markings (5, 6) is less than 90% of a thickness of the product (1).

3. The soft-chewable product (1) according to claim 2, wherein the two score markings (5, 6) have an identical depth, each of the score markings (5, 6) having a depth of between 10% and 45%, advantageously between 25% and 35%, of a thickness of the product (1).

4. The soft-chewable product (1) according to claim 1, having a friability of less than 0.5%, preferably less than 0.2%.

5. The soft-chewable product (1) according to claim 1, wherein:
the humectant is selected from the group consisting in propylene glycol, glyceryl triacetate, vinyl alcohol, neoagarobiose, sugar polyols such as glycerol, sorbitol, xylitol and maltitol, polymeric polyols such as polydextrose, caprylic/capric triglyceride, propylene glycol dicaprilate/dicarprate, medium chain triglycerides sold under the trademark Miglyol, especially Miglyol 812 or 814, vegetable oils, or mixtures thereof, and
the forming agent is selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline wax, cetyl alcohol, polysaccharides, hydroxypropylmethylcellulose, poly(meth)acrylates, alkyl poly(meth)acrylates, and mixtures thereof.

6. The soft-chewable product (1) according to claim 1, further comprising:
a filler selected from the group consisting in starch such as corn starch, rice starch or potato starch, sucrose, confectioner's sugar, lactose including anhydrous lactose, hydrated lactose, sprayed dried lactose, dextrin, dextrate, mannitol, soy grits, soy protein fines microcrystalline cellulose, silicified microcrystalline cellulose, calcium phosphate, crystalline maltose, maltodextrins and mixtures thereof, and
a palatant selected from the group consisting in animal product-based palatants such as dried or powdered meat and meat parts such as beef, pork, chicken, turkey, fish, and lamb; organ meats such as liver and kidney; meat meals, bone meals and ground bone and non-animal product based palatants such as essential oil, oleoresin, essence or extract, protein hydrolysate, distillate, or any product of roasting, heating or enzymolysis, which contains the flavoring constituents derived from a spice, fruit or fruit juice, vegetable or vegetable juice, edible yeast (active and inactive), herb, bark, bud, root, leaf or any other edible portions of a plant, and mixtures thereof.

7. The soft-chewable product (1) according to claim 1 for its use as a medicament.

8. The soft-chewable product (1) for its use according to claim 7, wherein the product is divided into doses to administer to the subject to be treated the appropriate quantity of active pharmaceutical, veterinary or nutraceutical substance.

9. The soft-chewable product (1) for its use according to claim 7, wherein the appropriate quantity of active pharmaceutical, veterinary or nutraceutical substance are administered at defined intervals.

10. The soft-chewable product (1) for use according to claim 7, said product being intended for a dog or a cat, preferably a dog.

11. The soft-chewable product (1) for use according to claim 10, wherein the active substance is a nutraceutical substance.

12. A process for preparing a soft-chewable product according to claim 1, comprising the steps of:
(a) obtaining a composition comprising:
a pharmaceutical, veterinary or nutraceutical active substance,
a humectant selected from the group consisting in propylene glycol, glyceryl triacetate, vinyl alcohol, neoagarobiose, sugar polyols such as glycerol, sorbitol, xylitol and maltitol, polymeric polyols such as polydextrose, caprylic/capric triglyceride, propylene glycol dicaprilate/dicarprate, medium chain triglycerides, vegetable oils, or mixtures thereof, and
a forming agent selected from the group consisting in polyvinylpyrrolidone, cross-linked polyvinylpyrrolidone, polyethylene glycols of various grades including PEG 3350, PEG 4000, PEG 6000 and PEG 8000; co-polymers of vinylpyrrolidone and vinyl acetate, polypropylene glycols, polyethylene glycol-polypropylene glycol copolymers (poloxamers), microcrystalline wax, cetyl alcohol, polysaccharides, hydroxypropylmethylcellulose, poly(meth)acrylates, alkyl poly(meth)acrylates, and mixtures thereof,
(b) forming the composition to obtain the product having score markings.

13. The process according to claim 12, wherein the forming of the composition takes place by compression or by molding, the score markings (5, 6) being hollowed in step (b).

14. The process according to claim 12, wherein the forming takes place by extrusion, the score markings (5, 6) being hollowed in step (b) through a die having a shape appropriate to hollow the score markings.
